SEQUENCE LISTING (4 sequences)

SEQ ID NO: 1 - DNA, 52 bp, Homo sapiens
tgagccccaggaactggagcgaaagtragatttgccccatgaggaaaagctg

SEQ ID NO: 2 - DNA, 60 bp, Homo sapiens
actctgagccccaggaactggagcgaaagtagatttgccccatgaggaaaagctgaactc

SEQ ID NO: 3 - DNA, 25 bp, Artificial Sequence
  Description of Artificial Sequence Synthetic primer
ggagcttgtgaacgtgtgtatgagt

SEQ ID NO: 4 - DNA, 22 bp, Artificial Sequence
  Description of Artificial Sequence Synthetic primer
ccccaggaactggagcgaaatt

(19) United States
(12) Patent Application Publication
(10) Pub. No.: US 2008/0171335 A1
(43) Pub. Date: Jul. 17, 2008

(54) METHOD FOR PERSONALIZED DIET DESIGN

(75) Inventors: Jose M. Ordovas, Framingham, MA (US); Dolores Corella, Valencia (ES); Chao-Qiang Lai, Belmont, MA (US)

Correspondence Address: DAVID S. RESNICK, 100 SUMMER STREET, NIXON PEABODY LLP, BOSTON, MA, 02110-2131, US

(73) Assignee: TRUSTEES OF TUFTS COLLEGE, Medford, MA (US)

(21) Appl. No.: 11/948,099

(22) Filed: Nov. 30, 2007

Related U.S. Application Data
(60) Provisional application No. 60/872,026, filed on Nov. 30, 2006.

(51) Int. Cl. C12Q 1/68

(52) U.S. Cl. 435/6

(57) ABSTRACT

The invention provides methods and kits for designing a diet with a desired fat content for an individual in need thereof to allow the individual to, for example, maintain or reduce healthy weight, manage diabetes, for example by managing weight or accommodate a food allergy. The method comprises determining whether the individual carries APOA5 −1131T and/or C allele or both or any allele in chromosome 11 that is in a tight linkage disequilibrium with said-alleles, wherein if the individual is a homozygote for APOA5 −1131T allele or an allele in tight linkage disequilibrium with APOA5 −1131T allele, the designing of a diet comprises reducing a total fat content of the diet below 30% of total calorie intake and/or reducing the amount of monounsaturated fatty acids in the diet under about 11% of total calorie intake. We surprisingly found that these methods and kits apply to both females and males and to a variety of ethnic backgrounds.

### CROSS-REFERENCE TO RELATED APPLICATIONS

The present application claims benefit-under 35 U.S.C. §119(e) of provisional application Ser. No. 60/872,026, filed Nov. 30, 2006, the content of which is herein incorporated by reference in its entirety.

### GOVERNMENT SUPPORT

This study was supported by National Heart, Lung, and Blood Institute contract N01-HC-25195 and grant HL-54776, and by contracts 53-K06-5-10 and 58-1950-9-01 from the US Department of Agriculture Research Service. The Government of the United States has certain rights in the invention.

### BACKGROUND

Obesity and being overweight are an increasing source of illness in the world. These conditions are not anymore only limited to developed countries. The serious illnesses that increased body weight makes people susceptible to include, but are by no means limited to, cardiovascular diseases, diabetes and diseases of the structural nature, such as arthritis, and other joint problems. Due to the multiple factors that are suspected and known to be involved in regulating body weight, it is difficult to design effective diets for individuals based solely on the traditional “eat less and exercise more” regime.

Thus, health professionals, such as dieticians, nurses, and medical doctors, encounter a daily the need for more assistance in advising their clients and designing diets such as weight-loss diets, and other diets that are directed to alleviating diseases or disorders that can be regulated using a special diet, such as diabetes, rheumatoid arthritis, inflammatory bowel disease, and food allergies.

Methods that would assist in personalized diet design to achieve the goal to increase health of individuals with diverse genetic makeup are needed.

### SUMMARY

Accordingly, we provide methods and kits to assist in personalized diet design. Particularly, we provide methods for directing a diet to a person to maintain or improve their health, for example, by controlling the weight of the individual. The methods comprise analysis of APOA5 single nucleotide polymorphism at location −1131 and based on the results determining if the individual should change the total fat and/or total monounsaturated fatty acid composition of their diet to maintain health or a healthy weight or to reduce weight. If the individual is a homozygote for allele T (or A in the opposite strand) in this locus, it will be important for that individual to reduce the total amount of fat in the diet to under 30% of total calorie or energy intake, typically calculated per day or per week. It will also be important for that individual to reduce the amount of monounsaturated fatty acids (MUFAs) to under 11% of total calorie or energy intake. Conversely, an individual heterozygous or homozygous for allele C (G in the opposite strand) in the same locus can include 30% or more of total fat or more than 11% of MUFAs in their diet, whether it be a weight or health-maintenance or weightless diet.

The methods are based on our discovery that carriers of the APOA5 gene variation −1131T, particularly the homozygous carriers of the APOA5 gene variation −1131T variation are more susceptible to increase in body mass index (BMI) than the non-carriers of the APOA5 −1131T allele when their dietary energy intake consists equal or more than about 30% of fat. We have also discovered that if equal or more than 11% of the total-energy intake consists of monounsaturated fatty acids (MUFA), carriers of APOA5 −1131T allele, particularly homozygous carriers, are more susceptible to increase in their BMI than are the individuals who are carriers of the more rare APOA5 −1131C allele.

We have also discovered that the APOA5 −1131C allele is associated with about 37% reduction in risk for being overweight when the individual's energy intake comprises equal or more than about 30% fat.

Accordingly, in one embodiment, we provide a method for designing a personalized diet, for example, a personalized diet that is directed to avoid increase in BMI or induce a decrease in BMI, wherein one determines the presence or absence of APOA5 −1131C allele or any allele that is in tight linkage disequilibrium with the APOA5 −1131C allele that is analyzed from a biological sample from a subject. If the individual does not carry the APOA5 −1131C allele or any allele that is in tight linkage disequilibrium with the APOA5 −1131C allele, then the diet for the individual will be designed so that less than about 30% of the total energy intake will be from fat.

In one embodiment, one first determines in the individual is in need of dietary intervention, specifically for weight management, such as weight loss and/or weight maintenance, management of diabetes or management of food allergies.

In one embodiment, if the individual does not carry the APOA5 −1131C allele or any allele that is in tight linkage disequilibrium with the APOA5 −1131C allele, then the diet for the individual will be designed so that less than about 11% of the total fat intake will be from MUFAs.

If the subject is found to carry one or two APOA5 −1131C alleles or allele that is in tight linkage disequilibrium with the APOA5 −1131C allele, then the diet can be designed to comprise equal or more than 30% of fat from the total energy intake. For example, such an individual would be a better candidate to lose weight using diets high in fat and protein than an individual who is homozygous for APOA5 −1131T allele.

In one embodiment, one determines from a biological sample from a subject, the presence or absence of APOA5 −1131T allele. In one embodiment, one determines the presence of absence of two APOA5 −1131T alleles, i.e. whether or not the subject is a homozygote for APOA5 −1131IT allele.

In one embodiment, one determines from a biological sample from a subject the APOA5 −1131T>C genotype.

One can determine or analyze the genotype or alleles using any known genotyping method. In one embodiment, one uses nucleic acid amplification before the analysis.

One can use any biological sample from an individual or subject in determining the genotype, so long as the biological sample comprises nucleic acids, such as DNA, for example genomic DNA or RNA.

In one embodiment, the diet is a diet directed to induce weight-loss in an overweight or obese individual or maintain a healthy weight.

In one embodiment, the diet is directed to alleviate a food allergy.

In one embodiment, the diet is directed to control diabetes.

The genotype determination can be performed by a third party and submitted with or without knowledge of the end use for the genotyping results to a provider, such as a health care provider or other individual who intends to provide personalized diet design.

In one embodiment, we provide a kit for personal or institutional use, wherein the kit provides tools to take a biological sample and send the sample for analysis. The kit further provides instructions for determining desirable dietary fat and/or MUFA content based upon the result of the genotyping results such that if one received a result of a genotype wherein one or two APOA5 −1131C alleles or any allele that is in tight linkage disequilibrium with the APOA5 −1131C allele, one can consider a diet, for example a weight-loss diet that derives equal or more than 30% of the total daily energy from fat or equal or more than 11% of daily energy from MUFAs. To the contrary, if one receives a result that indicates homozygosity for allele APOA5 −1131T, one should avoid diets that derive equal or more than 30% of daily energy from fat, or equal or more than 11% of daily energy from MUFAs.

In one embodiment, the kits and methods are directed to a mixed population, for example the U.S. population at large, and includes both male and female individuals. The individuals may be children, adolescents or adults.

In one embodiment, the kits and methods are directed to a population of Caucasian decent.

In one embodiment, the kits and methods are directed to a population of Northern European decent.

In one embodiment, the kits and methods are directed to a population of Mediterranean decent.

In one embodiment, the kits and methods are directed to a population of African-American decent.

In one embodiment, the invention provides 1. A method for directing a diet to an individual in need thereof to allow the individual to maintain or reduce weight, manage diabetes or accommodate a food allergy, the method comprising determining whether the individual carries APOA5-1311T and/or C allele or both or any allele in chromosome 11 that is in a tight linkage disequilibrium with said alleles, wherein if the individual is a homozygote for APOA5 −1131T allele or an allele in tight linkage disequilibrium with APOA5 −1131T allele, the individual is directed to a diet comprising a total fat content below 30% of total calorie intake and/or amount of monounsaturated fatty acids under about 11% of total calorie intake. In one embodiment the individual is Caucasian or African American.

In one embodiment, on further determines whether the individual carries at least one APOA5 −1131C allele or any allele in a fight linkage disequilibrium with said allele, and if the individual does not carry at least one APOA5 −1131C allele or any allele in a tight linkage disequilibrium with said allele, the individual is directed to a diet comprising a total fat content below 30% of total calorie intake and/or amount of monounsaturated fatty acids under about 11% of total calorie intake.

In one embodiment, the invention provides a kit for assisting an individual in determining whether a diet with total fat content 300% or more of total daily calorie intake or a diet with total monounsaturated fatty acid content of about 11% or more is suitable for the individual, the kit comprising a means to obtain a biological sample comprising nucleic acids, a packaging material for sending the biological material to be analyzed by a third party, optionally a return envelope for the third party to send a result of a to the individual or a requesting party, and an instruction leaflet which indicates that if the individual is a homozygote for the APOA5 −1131T allele or any acronym thereof, a diet with total fat content 30% or more of total daily calorie intake or a diet with total monounsaturated fatty acid content of about 11% or more is not suitable for the individual and if the individual carries one or two APOA5 −1131C alleles, the diet may be suitable for the individual.

In one embodiment, the kit further indicates that it is useful for individuals Caucasian and African American.

### BRIEF DESCRIPTION OF FIGURES

FIGS. 1A-1B show predicted values of body mass index (BM) by the −1131T>C (FIG. 1A) and the C56G polymorphisms (FIG. 1B) depending on the total fat consumed (as continuous) in both men and women. Predicted values were calculated from the regression models containing total fat intake, the corresponding APOA5 polymorphism, their interaction term, and the potential confounders (sex, age, tobacco, smoking, alcohol consumption, diabetes status, total energy intake, carbohydrate (as dichotomous), protein (as dichotomous), plasma triglycerides and familial relationships. P values for the interaction terms between fat intake (as continuous) and the corresponding APOA5 polymorphism were obtained in the hierarchical multivariate-interaction model containing total fat intake, the APOA5 SNP and additional control for the other covariates. Open symbols represent estimated values for wild-type homozygotes and solid symbols represent estimated values for the variant allele.

FIGS. 2A-2B show mean body mass index (BMI) in both men and women depending on the −1131 T>C polymorphism (FIG. 2A), or the C56G polymorphism (FIG. 2B) at the APOA5 gene according to the level of MUFA intake (below and above the population mean, 11% of energy). Estimated means were adjusted for sex, age, tobacco, smoking, alcohol consumption, diabetes status, total energy intake, carbohydrate (as dichotomous), protein (as dichotomous), plasma triglycerides and familial relationships. P values for the interaction terms between fat intake and the corresponding polymorphism were obtained in the hierarchical multivariate-interaction model containing MUFA intake as a categorical variable, the APOA5 SNP and additional control for the other covariates. Bars indicate standard error (SE) of means.

### DETAILED DESCRIPTION

We have found a consistent gene-diet interaction between the −1131T>C polymorphism in the APOA5 gene and total fat intake in determining obesity-related measures (BMI, overweight and obesity) in a large and heterogenous-US-population-based study.

Specifically, we found that higher n-6 (but not n-3) PUFA intake increased fasting triglycerides, remnant-like particle concentrations, and VLDL size and decreased LDL size in APOA5 −1131C minor allele carriers, but such interactions were not observed in carriers of the variant allele for the APOA5 56C>G polymorphism, suggesting different mechanisms driving the biological effects associated with these APOA5 gene variants or haplotypes (U.S. provisional application Ser. No. 60/60/717,345, filed on Sep. 15, 2005, the content of which is herein incorporated by reference in its entirety). Surprisingly, this association was equally present in both male and female populations and throughout wide selection of population background, such as Caucasian population in general, African American population, populations of Mediterranean origin as well as populations of Northern European origin.

This gene-diet interaction was not observed when we examined another genetic marker within the same gene, namely the 56C>G (S19W) polymorphism. Previous reports have demonstrated that these two SNPs are not in linkage disequilibrium (LD) and are considered two tag SNPs representing three APOA5 haplotypes (25, 26, 28). Although both SNPs have been associated with higher plasma triglyceride concentrations in several populations (25, 27, 28, 38-40), they appear to differ in their associations with other cardiovascular risk factors (26, 41). Moreover, in a recent report in the Framingham Heart Study (22) we have demonstrated gene-diet interactions between the APOA5 gene variation and PUFA intake in determining plasma fasting triglycerides, remnant lipoprotein concentrations, and lipoprotein particle size that were exclusive for the −1131T>C polymorphism.

Here we found that subjects homozygous for the −1131T, major allele, presented the expected positive association between fat intake and BMI. Conversely, in subjects carrying the APOA5 −1131C minor allele (−13% of this population), higher fat intakes were not associated with higher BMI. In contrast, this gene-fat interaction was not detected in carriers of the 56G minor allele. In these individuals, BMI increased as total fat intake increased following the same trend observed for subjects homozygous for the APOA5 56C major allele.

The −1131 site is defined to be 1131th nucleic acid promoter region 5′ from the origin of translation of the APOA5 (apolipoprotein A-V) gene. The APOA5 nucleic acid sequence for the purposes of defining the origin of translation can be found, for example, in GeneLoc location for GC11M116165 starting from 116,165,293 bp from pter of Chromosome 11, ending to 116,167,821 bp from pter. The gene is 2,528 bases in minus orientation. The accession No. for the APOA5 gene at GeneBank is AF202889. The gene is located proximal to the apolipoprotein gene cluster on chromosome 11q23. The reference sequence for the mRNA of the gene is NM_{—}052968.3.

The sequence around the polymorphism APOA5 −1131T/C is as follows: TGAGCCCCAGGAACTGGAGCGAAAGT[A/G]AGATTTGCCCCATGAGGAAAAGCTG (SEQ ID NO: 1), and can be found in dbSNP database with accession No. ss3199915 (see, e.g., Pennaccio et al. Ref. No. 23) or rs662799 or ss1943495, the sequence of which is as follows: actctgagcoccaggaactggagcgaaagt agatttgccccatgaggaaaagctgaactc (SEQ ID NO: 2).

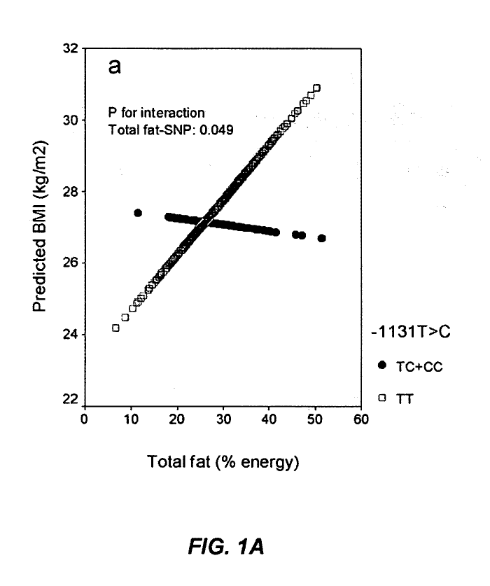

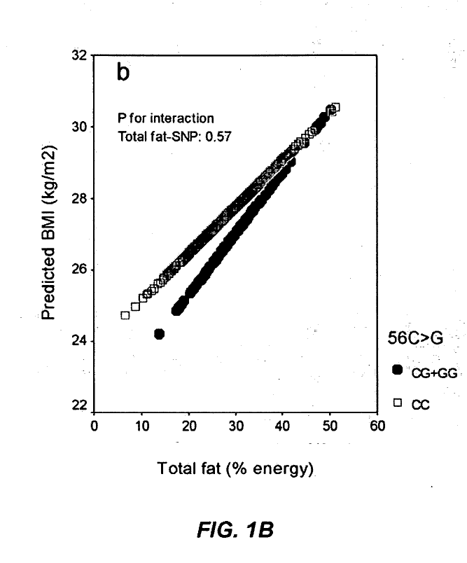

FIG. 1A of Talmud et. al. (24) shows the map of APOC3/A4/A5 gene cluster on chromosome 11p23 showing the position of the genes, direction of transcription and position of the variants that they studies, including the −1131T>C polymorphism.

The polymorphism can be analyzed, for example using the following protocol.

The following oligonucleotides were used for amplification as described by Talmud et al. (24): Forward primer 5′ GGAGCTTGTGAACGTGTGTATGAGT (SEQ ID NO: 3) and reverse primer 5′CCCCAGGAACTGGAGCGAAATT (SEQ ID NO: 4). This amplification is designed to force a C>A (T in the reverse primer), which introduced a Msel restriction site. These primers yield a PCR fragment of 154 bp which after restriction enzyme digestion products fragments of 133 bp and 21 bp for the T allele and a single uncut product for the C allele. For the use of these primers, the PCR conditions can be, for example, an initial denaturation of 96° C./5 mins followed by 30 cycles of 96° C./30 secs 60° C./30 secs, 72° C./30 secs, and a final extension period at 72° C./10 mins.

A skilled artisan knowing the sequence can easily design a variety of detection methods based on the known sequences around the polymorphism.

This gene-diet interaction between total fat intake and the −1131T>C polymorphism was consistently found whether fat intake was considered as a categorical or as a continuous variable. In addition, this interaction effect was homogenously found in both men and women adding support to its potential causal role.

Furthermore, when we considered BMT dichotomously to estimate the effect of this gene-diet interaction on obesity risk, we also found a statistically significant interaction between total fat intake and the APOA5 −1131T>C polymorphism. Our data revealed that in carriers of the −1131C minor allele a higher fat intake was not associated with a higher BMI, and thus we discovered a reduced obesity risk among −1131C minor allele carriers consuming a high-fat diet. We found ⅓ the risk of obesity in subjects carrying the −1131C minor allele compared with −1131T homozygotes only in the high category of total fat intake (>=30% of energy). Our population was varied and this association was not found to be related to gender or any particular sub-population of the U.S. based study population with varying ethnic background. Thus, individuals carrying the APOA5 −1131C allele are significantly protected from the health risks of high fat diets. In the low category of total fat intake (<30% energy from fat), the −1131C allele was not associated with a lower obesity risk. These results were consistently found when risk of overweight instead obesity was considered and no heterogeneity by sex was detected.

We are not aware of published studies focusing on reported interactions between dietary fat, the APOA5 −1131T>C SNP and BMI or obesity. To our knowledge, only one related paper reporting an association between the −1131T>C polymorphism and weight loss after short-term diet has been published (42). In this research, Aberle et al. (42) investigated how a short-term diet in a group of 606 hyperlipemic men from Hamburg affected BMI and lipid traits depending on the −1131T>C polymorphism. In their study, the investigators found no differences in BM1 at baseline between TT homozygotes and carries of the −1131C allele. However, following three months of energy restriction, patients with the −1131C. allele lost significantly more weight (13.4%) than did TT homozygotes (0.04%; P=0.002). This higher rate of weight loss in subjects carrying the −1131C allele is in agreement with our results indicating no increase in BMI with increase in total fat intake, and compatible with the hypothesis of Aberle et al (42), suggesting that the impaired ribosomal translation efficiency linked to the −1131C allele (43) may cause a reduced lipoprotein lipase-mediated triglyceride uptake into adipocytes and a more efficient decrease in BMI. In addition, Koike et al (32) have reported that over-expression of lipoprotein lipase significantly suppressed high fat diet-induced obesity and insulin resistance in transgenic Watanabe heritable hyperlipemia rabbits. Other potential mechanisms may involve a different regulation of the APOA5 gene by thyroid hormones (34) or PPARs (33) depending on the promoter allele and the fat intake. However, the design of our study cannot address the mechanisms by which dietary fat interacts with the −1131T>C polymorphism in determining BMI and further studies are needed.

We also found that only MUFA provided an interaction term that was statistically significant. However, in this U.S. population, MUFA and SFA are highly correlated (13). Therefore, studies in other populations consuming a Mediterranean type diet in which such correlation is lower are needed to confirm the specific benefit of a high-MUFA diet in carriers of the −1131T>C polymorphism. Moreover, despite the general consistency regarding the association of the APOA5 variant alleles with higher triglyceride concentrations, their relation with coronary artery disease remains highly controversial. Therefore, a careful investigation of this gene-diet interaction may help to explain these contradictory results with clinical outcomes (26, 40, 41, 44-47).

Based on our findings, carriers of the −1131C allele, despite their increase in plasma triglycerides, have a lower likelihood of obesity when consuming a high fat (specifically, high MUFA) diet as compared with subjects homozygous for the −1131T allele.

This circumstance may mask the risk estimation of cardiovascular disease if this interaction is not considered. Supporting this hypothesis are our recent results in the Framingham study (21) where we found that the association between the haplotype defined by the 56C>G polymorphism (for which no gene-fat interaction in determining obesity risk is present) was associated with higher carotid IMT compared with the wild-type haplotype, whereas the haplotypes defined by the presence of the rare allele in the −1131T>C, −3A>G, IVS+476G>A, and 1259T>C genetic variants were associated with higher carotid IMT only in obese subjects.

As used herein, a “tight linkage disequilibrium” means a polymorphic marker that co-segregates 100% with the allele “C” or “T” in the APOA5 −1131 locus. Linkage disequilibrium (LD) is a term used in the study of population genetics for the non-random association of alleles at two or more loci. Typically, if the alleles are in physical proximity with each others and one can see no recombination between the two alleles, they are called linked, and they are in 100% linkage disequilibrium with respect to each other. If both such alleles are polymorphic, either one of these polymorphic markers can be used in analysis of, for example a phenotype that has been found to be associated with one of the alleles. Thus, if one were to identify a marker that co-segregates 100% of the time with APOA5 −1131 allele T (or in its non-coding strand, allele A), such an allele can be easily substituted for the analysis of the of APOA5 −1131 allele T (or in its non-coding strand, allele A). A skilled artisan can easily calculate linkages between two alleles, for example, using the International HapMap Project which enables the study of LD in human populations online, for example, at World Wide Web address hapmap “dot” org.

The polymorphisms are analyzed from nucleic acids, for example isolated nucleic acids from any biological sample taken from an individual. Preferably one analyzed a sample that comprises genomic DNA. The sample may be directly analyzed or purified to varying degree prior to subjecting it to the genotype analysis.

Biological sample used as a source material for isolating the nucleic acids in the instant invention include, but are not limited to solid materials (e.g., tissue, cell pellets, biopsies, hair follicle samples buccal smear or swab) and biological fluids (e.g. blood, saliva, amniotic fluid, mouth wash, urine). Any biological sample from a human individual comprising even one cell comprising nucleic acid, can be used in the methods of the present invention.

The biological sample may be analyzed directly or stored before analysis.

Nucleic acid molecules of the instant invention include DNA and RNA, preferably genomic DNA, and can be isolated from a particular biological sample using any of a number of procedures, which are well-known in the art, the particular isolation procedure chosen being appropriate for the particular biological sample. Methods of isolating and analyzing nucleic acid variants as described above are well-known to one skilled in the art and can be found, for example in the Molecular Cloning: A Laboratory Manual, 3rd Ed., Sambrook and Russel, Cold Spring Harbor Laboratory Press, 2001.

The APOA5 polymorphisms according to the present invention can be detected from isolated-nucleic acids using techniques including direct analysis of isolated-nucleic acids such as Southern Blot Hybridization (DNA) or direct nucleic acid sequencing (Molecular Cloning: A Laboratory Manual, 3rd Ed., Sambrook and Russel, Cold Spring Harbor Laboratory Press, 2001). Some well known techniques do not require isolation of nucleic acids and such techniques are considered naturally to be part of the methods of the invention when analysis is performed from nucleic acids that have not been specifically isolated from the biological sample.

An alternative method useful according to the present invention for direct analysis of the APOA5 polymorphisms is the INVADER® assay (Third Wave Technologies, Inc (Madison, Wis.). This assay is generally based upon a structure-specific nuclease activity of a variety of enzymes, which are used to cleave a target-dependent cleavage structure, thereby indicating the presence of specific nucleic acid sequences or specific variations thereof in a sample (see, e.g. U.S. Pat. No. 6,458,535).

Preferably, a nucleic acid amplification, such as PCR based techniques are used. After nucleic acid amplification, the polymorphic nucleic acids can be identified using, for example direct sequencing with radioactively or fluorescently labeled primers; single-stand conformation polymorphism analysis (SSCP), denaturating gradient gel electrophoresis (DGGE); and chemical cleavage analysis, all of which are explained in detail, for example, in the Molecular Cloning: A Laboratory Manual, 3rd Ed., Sambrook and Russel. Cold Spring Harbor Laboratory Press, 2001.

The APOA5 polymorphisms are preferably analyzed: using methods amenable for automation such as the different methods for primer extension analysis. Primer extension analysis can be preformed using any method known to one skilled in the art including PYROSEQUENCING™ (Uppsala, Sweden); Mass Spectrometry including MALDI-TOF, or Matrix Assisted Laser Desorption Ionization—Time of Flight; genomic nucleic acid arrays (Shalon et al., Genome Research 6(7):639-45, 1996; Bernard et al., Nucleic Acids Research 24(8):1435-42, 1996); solid-phase mini-sequencing technique (U.S. Pat. No. 6,013,431, Suomalainen et al. Mol. Biotechnol. June; 15(2): 123-31, 2000); ion-pair high-performance liquid chromatography (Doris et al. J. Chromatogr. A May 8; 806(1):47-60, 1998); and 5′ nuclease assay or real-time RT-PCR (Holland et al. Proc Natl Acad Sci USA 88: 7276-7280, 1991), or primer extension methods described in the U.S. Pat. No. 6,355,433. Nucleic acids sequencing, for example using any automated sequencing system and either labeled primers or labeled terminator dideoxynucleotides can also be used to detect the polymorphisms. Systems for automated sequence analysis include, for example, Hitachi FMBIO® and Hitachi FMBIO® II Fluorescent Scanners (Hitachi Genetic Systems, Alameda, Calif.); Spectrumedix® SCE 9610 Fully Automated 96-Capillary Electrophoresis Genetic Analysis System (SpectruMedix LLC, State College, Pa.); ABI PRISM® 377 DNA Sequencer; ABI® 373 DNA Sequencer; ABI PRISM® 310 Genetic Analyzer; ABI PRISM® 3100 Genetic Analyzer, ABI PRISM® 3700 DNA Analyzer (Applied Biosystems, Headquarters, Foster City, Calif.); Molecular Dynamics FluorImager™ 575 and SI Fluorescent Scanners and Molecular Dynamics Fluorlmager™ 595 Fluorescent Scanners (Amersham Biosciences UK Limited, Little Chalfont, Buckinghamshire, England); GenomyxSC™ DNA Sequencing System (Genomyx Corporation (Foster City, Calif.); Pharmacia ALF™ DNA Sequencer and Pharmacia ALFexpress™ (Amersham Biosciences UK Limited, Little Chalfont, Buckinghamshire, England).

Nucleic acid amplification, nucleic acid sequencing and primer extension reactions for one nucleic acid sample can be performed in the same or separate reactions using the primers designed to amplify and detect the polymorphic APOA5 nucleotides.

In one embodiment, the invention provides a kit comprising one or more primer pairs capable of amplifying the APOA5 nucleic acid regions comprising the APOA5 −1131T>C alleles or alleles that are found to be in tight linkage disequilibrium with APOA5 −1131T>C polymorphic nucleotides of the present invention; buffer and nucleotide mix for the PCR reaction; appropriate enzymes for PCR reaction in same or separate containers as well as an instruction manual defining the PCR conditions, for example, as described in the Example below. The kit may further comprise nucleic acid probes to detect the APOA5 APOA5 −1131T>C alleles or alleles that are found to be in tight linkage disequilibrium with APOA5 −1131T>C. Primers may also be provided in the kit in either dry form in a tube or a vial, or alternatively dissolved into an appropriate aqueous buffer. The kit may also comprise primers for the primer extension method for detection of the specific APOA5 −1131T>C alleles or alleles that are found to be in tight linkage disequilibrium with APOA5 −1131T>Callelic polymorphism as described above.

The kit further provides instructions for an individual, individual provider or institutional provider regarding interpretation of the genotyping results. For example, the kit indicates that a presence of homozygosity for allele APOA5 −1131T is indicative of need for the individual who carries such genotype to reduce or maintain the amount of fat in the daily diet to be under 30% of the total energy intake and/or to reduce or maintain the amount of MUFAs to be under 11% of the total daily energy intake, if the individual wishes to maintain and/or reduce his/her BMI. For example, the kit may also include instructions that a homozygote, −1131 (T/T) alleles carrying individual should avoid weight-loss diets that have high fat content, such as higher than 30% or more of daily energy intake from fat or higher than about 11% of daily energy content from MUFAs. The kit may also include charts for individuals or dieticians to determine how much fat is 30% or more or under 30%, or how much MUFAs is about 11%, of their daily intake, or calculation advise to that extent. Typically, a skilled dietician will be able to design a diet, such as a weight loss or weight maintenance diet with fat content under about 30%.

The kit may further include a list of MUFAs that, for example, one may wish to avoid if one is an APOA5 −1131T homozygote. Such list may include fat sources including

Such instructions are an integral part of the kit because without such instructions, one can not interpret and thus benefit from the genotype analysis.

One may also combine the analysis of the present methods with any other genetic analysis to determine susceptibility to diseases or responses to certain nutrients such as polyunsaturated fatty acids.

### EXAMPLES

Genetic variability has been reported for all the identified candidate lipid-related genes; however, associations between many of these variants and lipid profiles have been highly controversial (1-4). One of the most accepted arguments to explain the lack of replication among studies has been the existence of gene-environment interactions (5-7). Overall, gene-environment interaction refers to the differential phenotypic effects of diverse environments on individuals with the same genotype or to the discrepant effects of the same environment on individuals with different genotypes (5-8).

The investigation of gene-environment interactions will assist in increasing replication among studies and consequently, in facilitating cardiovascular disease prevention. Nutrition is part of every individual from conception to death. Therefore, it is considered one of the most important environmental factors interacting with our genes to increase or decrease the likelihood of developing lipid disorders and further cardiovascular risk (9-11).

Currently, there are an increasing number of published studies reporting gene-diet interactions in relation to lipid metabolism (12). Among dietary factors, total fat, specific fatty acids, alcohol, carbohydrate and total energy intake have been the most studied (13-17). On the other hand and directly related to nutrition, obesity has been another factor widely reported to modulate genetic effects on lipid metabolism and cardiovascular risk (18-20).

The apolipoprotein A5 (APOA5) gene is an example of recently reported gene-diet and gene-obesity interactions (21, 22). In the Framingham Heart Study, we reported a gene-diet interaction between APOA5 gene variation and polyunsaturated fatty acids (PUFA) in relation to plasma lipid concentrations and lipoprotein particle size (21). Furthermore, we demonstrated that obesity modulates the effect of APOA5 gene variation in carotid intimal medial thickness (IMT), a surrogate measure of atherosclerosis burden. This association remained significant even after adjustment for triglycerides (22). APOA5 gene variation has been associated with increased triglyceride concentrations (23-27). Five common APOA5 single-nucleotide polymorphisms (SNPs) have been reported in several populations: −1131T>C, −3A>G, 56C>G IVS3+476G>A and 1259T>C (24-27). With the exception of the 56C>G SNP, the SNPs are reported to be in significant linkage disequilibrium (25-28). Moreover, the −1131T-C and the 56C>G (S19W) are considered tag SNPs, representing three APOA5 haplotypes.

The precise mechanism by which APOA5 influences plasma triglycerides and related-measures remains to be determined (29). However, activation of lipoprotein lipase has been suggested as one of the potential APOA5 hypotriglyceridemic mechanisms (30). Lipoprotein lipase has also been implicated in the development of obesity (31-32) and so are some of the APOA5 gene regulators (i.e., peroxisome proliferator-activated receptors (PPARs), insulin, thyroid hormones (33-34)).

### Subjects and Study Design

The study sample consisted of 2,280 subjects who participated in the Framingham Offspring Study (FOS) (35). Anthropometric, clinical and biochemical variables as well as dietary intake and other lifestyle variables were recorded for subjects who participated in the fifth examination visit conducted between 1992 and 1995 (n=3515). DNA was obtained during 1987-1991. The Institutional Review Board for Human Research at Boston University and Tufts University/New England Medical Center approved the protocol of the study reported here. All participants provided written informed consent. Only subjects with phenotypic data and complete dietary information for whom APOA5 gene variants were examined were included in this study. In addition, subjects with any missing data regarding control variables (age, BMI, tobacco smoking, alcohol consumption and diabetes status) were excluded from our analyses. Thus, data for 1073 men and 1207 women who met the above criteria were analyzed. Because nearly all subjects were non-Hispanic White, no control for ethnicity was needed. Although in the Framingham Study recruitment of families was planned (35), in this specific sample most participants were unrelated, and the number of individuals within each family included in the present study was very low. Thus, participants were distributed in 1483 pedigrees, of which 83% were singletons. In the non-singletons, most participants were siblings and cousins. Alcohol consumption was calculated in g/day based on the reported alcoholic beverages consumed in the previous year for each individual, and subjects were classified as non-drinkers (those who did not report consumption of alcohol), and drinkers (15). Smokers were defined as those who smoked at least 1 cigarette Id. Physical activity was assessed as a weighted sum of the proportion of a typical day spent sleeping and performing sedentary, slight, moderate or heavy physical activities. Subjects were classified as having type 2 diabetes if they were on hypoglycemic drug therapy for diagnosed type 2 diabetes at any study examination, of if they had fasting plasma glucose levels of at least 7.0 mmol/liter at two or more exams (36).

### Anthropometric and Biochemical Determinations

Height and weight were measured with the individuals dressed in an examining gown and wearing no shoes (19). BMI was calculated as weight in kilograms divided by the square of height in meters. Obesity was defined as BMI 30 kg/m² and overweight as BMI 25 kg/m². According to these international criteria, there were 550 obese (288 men and 262 women) and 1,507 overweight (854 men and 653 women) subjects in our study population. Fasting venous blood samples were collected and plasma was separated from blood cells by centrifugation and immediately used for the measurement of lipids. Fasting glucose, plasma lipids and lipoproteins were measured as previously described (16, 26, 36).

### Dietary Information

Dietary intake was estimated with a semiquantitative food-frequency questionnaire, described and validated by Rimm et al (37). This questionnaire includes 126 food items with specified serving size. Subjects were asked to report their frequency of use of each item per day, week or month over the past year by checking 1 of the 9 frequency categories. The mean daily intake of nutrients was calculated by multiplying the frequency of consumption of each item by its nutrient content per serving and totaling the nutrient intake for all food items. The Harvard University Food Composition Database, derived from US Department of Agriculture sources and supplemented with manufacturer information, was used to calculate nutrients and total energy intake. Macronutrient intake data were obtained in terms of absolute amounts (g/d). We modeled the effect of macronutrients in terms of nutrient density, i.e., the ratio of energy from the corresponding macronutrient to total energy, expressed as a percentage. Intakes of carbohydrate, protein, total fat, saturated fatty acids (SFA), monounsaturated fatty acids (MUFA) and total PUFA were calculated for each individual. These measures were included in the analyses as both continuous and categorical variables.

### Genetic Analyses

DNA was isolated from blood samples using DNA blood Midi kits (Qiagen, Hilden, Germany) according to the vendor's recommended protocol. The −1131T>C and the 56C>G SNPs at the APOA5 locus were determined using the ABI Prism SNapShot multiplex system (Applied Biosystems, Foster City, Calif.). The primers and probes used for genotyping were described previously (25). Standard laboratory practices such as blinded replicate samples and positive and negative controls were used to ensure the accuracy of genotype data.

### Statistical Analyses

We examined all continuous variables for normality of distribution. Triglyceride concentrations were log transformed. Pearson χ2 and Fisher tests were used to test differences between observed and expected genotype frequencies, assuming Hardy-Weinberg equilibrium, and to test differences in percentages. The pair-wise linkage disequilibria (LDs) between SNPs at the APOA5 locus were estimated with the coefficient R2, with the HelixTree program. Due to the low frequency of the variant alleles, carriers and non-carriers of the minor allele for each polymorphism were grouped and compared with wild-type homozygotes. T tests were applied to compare crude means. The relationships between APOA5 genotype, dietary macronutrient intake, and BMI were evaluated by analysis of covariance techniques and adjusted means were estimated. In these analyses, we used several different models to test the consistency of results and to adjust for potential confounders. Dietary macronutrient intakes were included in the analyses as both continuous and categorical variables. To construct the categorical variables, intakes were classified into two groups divided by the mean value of the population. Interactions between dietary macronutrients (as categorical or as continuous variables) and the APOA5 polymorphisms were tested in a hierarchical multivariate-interaction model after controlling for potential confounders, including sex, age, smoking, alcohol consumption, total energy intake and diabetes status. Additional control for the other macronutrients and for plasma triglyceride concentrations were carried out.

Because the present study involved some correlated data that were due to familial relationships (siblings and cousins), we also controlled for familial relationships. We used two approaches to accomplish these analyses. First, a generalized linear mixed-model approach, which assumed an exchangeable correlation structure among all members of a family (PROC MIXED in SAS, Cary, N.C.), was used. Second, because this approach could not accurately represent the true correlation structure within these pedigrees, we used a measured-genotype approach as implemented in SOLAR, a variance component-analysis computer package for quantitative traits measured in pedigrees of arbitrary size. After having checked that the results obtained using the generalized mixed model were similar to those of the SOLAR approach because of the large number of unrelated subjects in this sample, we decided to present data obtained with the generalized mixed model for the adjustment of familial relationships. Statistical analyses were performed for the whole sample and for men and women separately in order to evaluate the homogeneity of the effect. Standard regression diagnostic procedures, including multicollinearity tests, homogeneity of variance tests and normal plots of the residuals, were used to ensure the appropriateness of these models. When total fat intake was considered as a continuous variable, its interaction with the corresponding APOA5 polymorphism was depicted by computing the predicted values for each individual from the adjusted regression model and plotting these values against fat intake by APOA5 genotype.

For a dichotomous outcome, obesity was defined as BMI≧30 kg/m² and overweight BMI≧25 kg/m². Logistic regression models were fitted to estimate the odds ratio (OR) and 95% confidence interval (CI) of obesity and overweight associated with the presence of each genetic variant as compared with the wild-type. These multiple logistic regression models were also fitted to control for the effect of covariates and familial relationships and to test the statistical significance of the corresponding gene-diet interaction terms. Statistical analyses were carried out using SAS software. All reported probability tests were two-sided.

### Results

Table 1 displays demographic, anthropometric, clinical, biochemical, lifestyle and genetic characteristics of the studied population. Genotype frequencies did not deviate from Hardy-Weinberg equilibrium expectations. Pair-wise LD coefficient R between the −1131T>C and 56C>G SNPs was 0.063, indicating the haplotypic independence of both markers. Neither the −1131T>C nor the 56C>G SNPs were statistically associated with BMI in crude analyses (P=0.73; P=0.58, respectively) or after control for potential confounders. Then we examined if macronutrient intake modulates the association between these polymorphisms and BMI in the whole population. As a first approach we examined the effect of macronutrients as categorical variables. Total fat, carbohydrate and protein intakes were classified into two groups according, to the mean value of the population (30%, 50% and 15%, respectively). We found a gene-diet interaction between the −1131T>C polymorphism and fat intake in relation to BMI (P=0.001), that remained statistically significant after controlling for sex, age, alcohol consumption, tobacco smoking, physical activity, diabetes status, total energy, protein and carbohydrate intake (P=0.018) and after additional adjustment of this multivariate model for familial relationships (0.047). Further adjustment for physical activity index did not modify the significance of the results for this and all the subsequent models. This interaction was not found for carbohydrate intake or for protein intake neither in the crude model nor in the multivariate model adjusted for familial relationships (P=0.39 and P=0.56, respectively).

Table 2 shows BMI and P values for men and women combined depending on the amount of the macronutrient consumed in the diet and the APOA5 polymorphism. The interaction effect of the −1131T>C polymorphism with total fat on BMI revealed that the increase in BMI associated with a higher fat intake (>=30% of energy from fat) observed in subjects homozygotes for the −1131T major allele was not present in carriers of the −113C minor allele at the APOA5 locus (−13% of the population). This interaction was not observed for the 56C>G polymorphism (P=0.55). Taking into account that APOA5 polymorphisms have been associated with triglycerides in several studies, an additional adjustment for plasma triglyceride concentrations was carried out. After this additional adjustment, the gene-diet interaction between the −1131T>C SNP and total fat intake in determining BMI remained statistically significant (P=0.044).

Moreover, there was no evidence that the effect of this interaction differed between men and women (P for heterogeneity by gender=0.477). Likewise, no heterogeneity by sex was observed in the no interaction between the 56C>G polymorphism and total fat intake as dichotomous on BMI (P=0.985).

To explore a possible dose-response relationship in the −1131T>C-fat interaction and to avoid the problem of selection of cut-off points, total fat intake was considered as a continuous variable. As no heterogeneity of the effect by sex was observed (P for interaction with sex=0.93), subsequent analyses combined men and women and the model additionally adjusted for sex.

In agreement with the data obtained using total fat as a qualitative variable, the modification of the effect of the −1131T>C polymorphism by total fat intake appeared to be linear in determining BMI (FIG. 1a). After adjustment for covariates, including plasma triglyceride concentrations, a statistically significant interaction (P=0.048) between total fat intake as a continuous variable and the −1131T>C polymorphism in determining BMI was found.

Thus, in subjects homozygous for the −1131T allele, BMI increased as total fat intake increased. In contrast, among carriers of the −1131C allele, the expected increase was not observed and those with higher fat intake appeared to have lower BMI. On the other hand, no significant interaction between the 56C>G polymorphism and total fat (P=0.57) was detected when the same regression model was fitted (FIG. 1b). In both wild-type and carriers of the variant allele, BMI increased as total fat intake increased.

Furthermore, we examined the effect of specific fatty acids on the interaction between the −1131T>C polymorphism and fat in relation to BMI. When each fatty acid intake was examined separately as a dichotomous variable, by population mean (10% energy for SFA, 11% for MUFA as 6% for PUFA), although the direction of the effect was similar for each of these fatty acids, only the interaction between the −1131T>C polymorphism and MUFA intake reached statistical significance. (P=0.024 in the multivariate model adjusted for sex, age, tobacco smoking, alcohol consumption, diabetes status, total energy, protein, carbohydrate, plasma triglycerides and familial relationships). No significant interactions between specific fatty acids and the 56C>G polymorphism on BMI were detected. No statistically significant heterogeneity by sex was detected neither for the −1131T>C nor for the 56C>G polymorphisms.

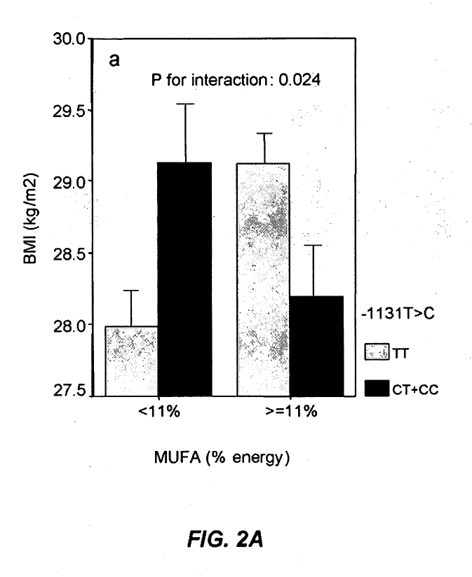

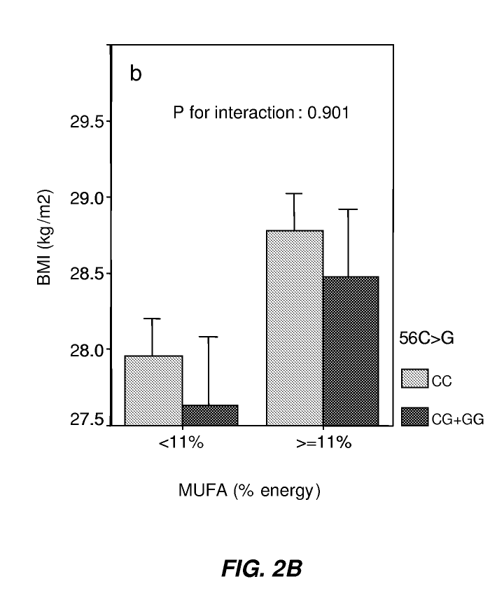

FIG. 2 shows mean BMI depending on the −1131T>C polymorphism (a) or the 56C>G polymorphism (b) and the MUFA intake in men and women. Finally, the effect of the APOA5-fat interaction on the obesity risk was examined. There were 550 obese subjects and 1730 non-obese. After adjustment for sex, age, tobacco smoking, alcohol consumption, diabetes, total energy intake, protein, carbohydrate, plasma triglycerides and familial relationships, we found a statistically significant interaction between the −1131>C polymorphism and total fat intake as a dichotomous variable (less or more than 30%) in obesity risk (P=0.049). No statistically significant interaction was found for the 56C>G polymorphism (P=0.24) when the same logistic regression model was fitted. A stratified analysis by fat intake (Table 3) clearly provides evidence of the gene-diet interaction effect between the −1131T>C polymorphism and total fat intake in determining the risk of obesity. When the level of fat intake was low (<30% of energy), subjects with the −1131C allele had a non-significant modest increase in obesity risk. However, in subjects consuming >=30% of energy from fat, carriers of the −1131C allele have about ⅓ the risk of obesity (OR: 0.61, 95% CI: 0.39-0.98; P=0.032)) compared with the −1131T homozygotes.).

When the specific fatty acids were analyzed we observed a statistically significant interaction (P=0.026) between MUFA intake and the −1131C>T polymorphism on obesity risk after adjustment for sex, age, tobacco smoking, alcohol consumption, diabetes, total energy intake, protein, carbohydrate, plasma triglycerides and familial relationships. Thus, in subjects consuming >=11% of energy from MUFA, carriers of the −1131C allele have 38.2% lower obesity risk (OR: 0.62, 95% CI: 0.41-0.94; P=0.026) compared with the −1131T homozygotes. No heterogeneity of this effects by sex was observed (P for interaction sex-genotype-fat>0.05.

Moreover, when the risk of being overweight was studied (1507 overweight and 773 non-overweight subjects), we also obtained a significant interaction between the −1131T>C polymorphism and total fat intake, that remained statistically significant after adjustment for sex, age, tobacco smoking, alcohol consumption, diabetes, total energy intake, protein, carbohydrate, plasma triglycerides and familial relationships (P=0.029).

Table 3 shows OR estimations of overweight for the −1131T>C polymorphism in the stratified analyses by total fat intake. In line with the previous results concerning obesity risk the −1131C minor allele was associated with a 37% reduction of overweight risk (P=0.031) in subjects consuming >=30% of energy from fat when compared with TT homozygotes. No reduction of overweight risk in carriers of the −1131C minor allele was found if the level of total fat intake was lower. No statistically significant interaction between total fat intake and the 56C>G polymorphism in determining the risk of overweight was found (P=0.79). No statistical significant heterogeneity by sex in the tested interactions on overweight risk was detected.

TABLE 1
Demographic, biochemical, dietary and genotypic characteristics
of participants according to gender

| | MEN (n = 1073) | | WOMEN (n = 1207) | |
|---|---|---|---|---|
| | Mean (SD) or n(%) | | Mean (SD) or n(%) | |
| Age (years) | 54.5 | (9.8) | 53.9 | (9.6) |
| Body mass index (kg/m²) | 28.21 | (4.0) | 26.72 | (5.5) |
| Total-cholesterol (mg/dL) | 202 | (34) | 208 | (37) |
| LDL-C (mg/dL) | 129 | (22) | 124 | (23) |
| HDL-C (mg/dL) | 43 | (11) | 56 | (15) |
| Triglycerides (mg/dL) | 161 | (129) | 134 | (94) |
| Glucose (mmol/L) | 103 | (28) | 97 | (26) |
| Total Energy intake (kcal/day) | 2004 | (649) | 1726 | (575) |
| Total fat intake (% energy) | 29.8 | (6.3) | 29.2 | (6.3) |
| SFA (% energy) | 10.6 | (2.9) | 10.4 | (2.9) |
| MUFA (% energy) | 11.4 | (2.6) | 10.9 | (2.6) |
| PUFA (% energy) | 5.8 | (1.7) | 6.0 | (1.7) |
| Carbohydrates (% of energy) | 49.9 | (8.4) | 51.9 | (8.3) |
| Protein (% of energy) | 14.6 | (3.6) | 15.8 | (3.8) |
| Fiber (g/d) | 19.1 | (8.5) | 18.9 | (8.2) |
| Alcohol (g/d) | 3.7 | (4.6) | 1.8 | (2.6) |
| Drinkers (n, %) | 833 | (77.6) | 842 | (69.8) |
| Smokers (n, %) | 186 | (17.3) | 223 | (18.5) |
| Diabetic subjects (n, %) | 110 | (10.2) | 77 | (6.4) |
| Obese subjects (BMI >= 30 kg/m²) | 288 | (26.8) | 262 | (21.7) |
| Overweight subjects (BMI >= 25 kg/m²) | 854 | (79.6) | 653 | (54.1) |
| APOA5 −1131T > C, n (%) | | | | |
| TT | 877 | (86.6) | 936 | (87.7) |
| C carriers | 144 | (13.4) | 148 | (12.3) |
| APOA5 56C > G, n (%) | | | | |
| CC | 927 | (88.5) | 1058 | (89.4) |
| G carriers | 123 | (11.5) | 128 | (10.6) |

Values are listed as mean (standard deviation, SD) or as number (n) and percent (%)
LDL-C, low-density lipoprotein-cholesterol); HDL-C, high-density lipoprotein-cholesterol
SFA, saturated fatty acids; MUFA, monounsaturated fatty acids; PUFA, polyunsaturated fatty acids.

TABLE 2
Body mass Index (mean and standard error) depending on the amount
of macronutrient consumed in the diet and the APOA5 polymorphism

| | −1131T > C | | | 56C > G (S19W) | | |
|---|---|---|---|---|---|---|
| | TT (n = 1866) | TC + CC (n = 292) | P* for interaction | CC (n = 1985) | CG + GG (n = 251) | P* for interaction |
| APOA5 genotypes | Mean (SE) | Mean (SE) | APOA5-nutrient | Mean (SE) | Mean (SE) | APOA5-nutrient |
| Total fat | | | | | | |
| <30% energy | 27.09 (0.22) | 28.07 (0.47) | 0.047 | 27.22 (0.21) | 26.66 (0.49) | 0.552 |
| >=30% energy | 28.17 (0.21) | 27.01 (0.49) | | 27.97 (0.20) | 27.91 (0.48) | |
| Total carbohydrate | | | | | | |
| <50% energy | 28.47 (0.22) | 28.07 (0.50) | 0.385 | 28.36 (0.22) | 28.07 (0.51) | 0.645 |
| >=50 energy | 26.92 (0.21) | 27.28 (0.45) | | 27.16 (0.21) | 26.69 (0.48) | |
| Protein | | | | | | |
| <15% energy | 27.07 (0.21) | 27.35 (0.55) | 0.561 | 27.14 (0.21) | 26.78 (0.49) | 0.995 |
| >=15% energy | 28.35 (0.22) | 28.13 (0.51) | | 28.31 (0.22) | 28.03 (0.51) | |

SE: Standard error.
*P value obtained in the multivariate model for interaction after adjustment for age, sex, tobacco, smoking, alcohol consumption diabetes status, total enery intake and the other macronutrients as dichotomous (total fat, carbohydrates or proteins depending on the nutrient considered) and familial relationships

TABLE 3
Risk of obesity and risk of overweight depending on the −1131T > C
polymorphism and total fat intake in men and women

| | Total fat | | | | | | |
|---|---|---|---|---|---|---|---|
| | <30% energy | | | >=30% energy | | | P** for interaction |
| Phenotype | OR | 95% Cl | P* | OR | 95% Cl | P* | APOA5-Total fat |
| Obesity Risk | | | | | | | |
| TT | 1 | | | 1 | | | |
| TC + CC | 1.16 | (0.77-1.74) | 0.470 | 0.61 | (0.39-0.96) | 0.032 | 0.049 |
| Overweight risk | | | | | | | |
| TT | 1 | | | 1 | | | |
| TC + CC | 1.15 | (0.78-1.71) | 0.407 | 0.63 | (0.41-0.96) | 0.031 | 0.029 |

*P value for the genotype obtained in the corresponding logistic regression model after adjustment for age, sex, tobacco smoking, alcohol consumption, diabetes, total enery intake, protein (as dichotomous), carbohydrate (as dichotomous), plasma triglycerides and familial relationships in the corresponding strata of total fat consumption
**P value for the interaction term obtained in the corresponding logistic regression model for interaction after adjustment for sex, age, tobacco smoking, alcohol consumption, diabetes, total enery intake, protein (as dichotomous), carbohydrate (as dichotomous), plasma triglycerides and familial relationships

The references cited throughout the specification including the examples below are herein incorporated by reference in their entirety to the extent not inconsistent with the specification.

### REFERENCES

1. Cambien F. Coronary heart disease and polymorphisms in genes affecting lipid metabolism and inflammation (2005) Curr Atheroscler Rep 7:188-95.
2. Lai C Q, Parnell L D, Ordovas J M. The APOA1/C3/A4/A5 gene cluster, lipid metabolism and cardiovascular disease risk (2005) Curr Opin Lipidol 16:153-66.
3. Pajukanta P (2004) Do DNA sequence variants in ABCA1 contribute to HDL cholesterol levels in the general population? J Clin Invest 114(9):1244-7.
4. Semple R K, Chatterjee V K, O'Rahilly S (2006) PPAR gamma and human metabolic disease. J Clin Invest 116:581-9.
5. Yang Q, Khoury M J (1997) Evolving methods in genetic epidemiology. III. Gene-environment interaction in epidemiologic research. Epidemiol Rev 19:3343.
6. Khoury M J, Davis R, Gwinn M, Lindegren M L, Yoon P. Do we need genomic research for the prevention of common diseases with environmental causes? (2005) Am J Epidemiol 161:799-805.
7. Hunter D J (2005) Gene-environment interactions in human diseases. Nat Rev Genet 6:287-98.
8. Grigorenko E L (2005) The inherent complexities of gene-environment interactions. J Gerontol B Psychol Sci Soc Sci 60:53-64.
9. Ordovas J M, Corella D (2004) Nutritional genomics. Annu Rev Genomics Hum Genet 5:71-118.
10. Mutch D M, Wahli W, Williamson G (2005) Nutrigenomics and nutrigenetics: the emerging faces of nutrition. FASEB J 19:1602-16.
11. Afman L, Muller M (2006) Nutrigenomics: from molecular nutrition to prevention of disease. J Am Diet Assoc 106:569-76.
12. Corella D, Ordovas J M (2005) Single nucleotide polymorphisms that influence lipid metabolism: Interaction with Dietary Factors. Annu Rev Nutr 25:341-90.
13. Ordovas J M, Corella D, Demissie S, Cupples L A, Couture P, Coltell O, Wilson P W, Schaefer E J, Tucker K L (2002) Dietary fat intake determines the effect of a common polymorphism in the hepatic lipase gene promoter on high-density lipoprotein metabolism: evidence of a strong dose effect in this gene-nutrient interaction in the Framingham Study. Circulation 106:2315-21.
14. Dwyer J H, Allayee H, Dwyer K M, Fan J, Wu H, Mar R, Lusis A J, Mehrabian M (2004) Arachidonate 5-lipoxygenase promoter genotype, dietary arachidonic acid, and atherosclerosis. N Engl 3 Med 350:29-37.
15. Corella D, Tucker K, Lahoz C, Coltell O, Cupples L A, Wilson P W, Schaefer E J, Ordovas J M (2001) Alcohol drinking determines the effect of the APOE locus on LDL-cholesterol concentrations in men: the Framingham Offspring Study. Am J Clin Nutr 73:73645.
16. Memisoglu A, Hu F B, Hankinson S E, Manson J E, De Vivo I, Willett W C, Hunter D J (2003) Interaction between a peroxisome proliferator-activated receptor gamma gene polymorphism and dietary fat intake in relation to body mass. Hum Mol Genet 12:2923-9.
17. Miyaki K, Sutani S, Kikuchi H. Takei L Murata M, Watanabe K, Omae K (2005) Increased risk of obesity resulting from the interaction between high energy intake and the Trp64Arg polymorphism of the beta3-adrenergic receptor gene in healthy Japanese men. J Epidemiol 15:203-10.
18. Marques-Vidal P, Bongard V, Ruidavets J B, Fauvel J, Hanaire-Broutin H, Perret B, Ferrieres J (2003) Obesity and alcohol modulate the effect of apolipoprotein E polymorphism on lipids and insulin. Obes Res 11:1200-6.
19. Elosua R, Demissie S, Cupples L A, Meigs J B, Wilson P W, Schaefer E J, Corella D, Ordovas J M (2003) Obesity modulates the association among APOE genotype, insulin, and glucose in men. Obes Res 11:1502-8.
20. Corella D, Ordovas J M (2004) The metabolic syndrome: a crossroad for genotype-phenotype associations in atherosclerosis. Curr Atheroscler Rep 6: 186-96.
21. Elosua R, Ordovas J M, Cupples L A, Lai C Q, Demissie S, Fox C S, Polak J F, Wolf P A, D'Agostino R B Sr, O'donnell C J (2006) Variants at the APOA5 locus, association with carotid atherosclerosis, and modification by obesity: the Framingham Study. J Lipid Res 47:9906.
22. Lai C Q, Corella D, Demissie S, Cupples L A, Adiconis X, Zhu Y, Parnell L D, Tucker K L, Ordovas J M (2006) Dietary intake of n-6 fatty acids modulates effect of apolipoprotein A5 gene on plasma fasting triglycerides, remnant lipoprotein concentrations, and lipoprotein particle size: the Framingham Heart Study. Circulation 113:2062-70.
23. Pennacchio L A, Olivier M, Hubacek J A, Cohen J C, Cox D R, Fruchart J C, Krauss R M, Rubin E M (2001) An apolipoprotein influencing triglycerides in humans and mice revealed by comparative sequencing. Science 294:169-73.
24. Talmud P J, Hawe E, Martin S, Olivier M, Miller G J, Rubin E M, Pennacchio L A, Humphries SE (2002) Relative contribution of variation within the APOC3/A4/A5 gene cluster in determining plasma triglycerides. Hum Mol Genet 11:3039-46.
25. Lai C Q, Tai E S, Tan C E, Cutter J, Chew S K, Zhu Y P, Adiconis X, Ordovas J M (2003) The APOA5 locus is a strong determinant of plasma triglyceride concentrations across ethnic groups in Singapore. J Lipid Res 44:2365-73.
26. Lai C Q, Demissie S, Cupples L A, Zhu Y, Adiconis X, Parnell L D, Corella D, Ordovas J M (2004) Influence of the APOA5 locus on plasma triglyceride, lipoprotein subclasses, and CVD risk in the Framingham Heart Study. J Lipid Res 45:2096-105.
27. Hodoglugil U, Tanyolac S, Williamson D W, Huang Y, Mahley R W (2006). Apolipoprotein A-V: a potential modulator of plasma triglyceride levels in Turks. J Lipid Res 47:144-53.
28. Pennacchio L A, Olivier M, Hubacek J A, Krauss R M, Rubin E M, Cohen J C. Two independent apolipoprotein A5 haplotypes influence human plasma triglyceride levels (2002) Hum Mol Genet 11:3031-8.
29. Merkel M, Heeren J. Give me A5 for lipoprotein hydrolysis! (2005) J Clin Invest 115:2694-6.
30. Merkel M, Loeffier B, Kluger M, Fabig N, Geppert G, Pennacchio L A, Laatsch A, Heeren J (2005) Apolipoprotein AV accelerates plasma hydrolysis of triglyceride-rich lipoproteins by interaction with proteoglycan-bound lipoprotein lipase. Biol Chem 280:21553-60.
31. Mead J R, Irvine S A, Ramji D P (2002) Lipoprotein lipase: structure, function, regulation, and role in disease. J Mol Med 80:753-69.
32. Koike T, Liang J, Wang X, Ichikawa T, Shiomi M, Liu G, Sun H, Kitajima S, Morimoto M, Watanabe T, Yamada N, Fan J (2004) Overexpression of lipoprotein lipase in transgenic Watanabe heritable hyperlipidemic rabbits improves hyperlipidemia and obesity. J Biol Chem 279:7521-9.
33. Prieur X, Coste H, Rodriguez J C (2003) The human apolipoprotein AV gene is regulated by peroxisome proliferator-activated receptor-alpha and contains a novel farnesoid X-activated receptor response element. J Biol Chem 278:25468-80.
34. Prieur X, Huby T, Coste H, Schaap F G, Chapman M J, Rodriguez J C (2005) Thyroid hormone regulates the hypotriglyceridemic gene APOA5. J Biol Chem 280:2753343.
35. Feinleib M, Kannel W B, Garrison R J, McNamara P M, Castelli W P (1975) The Framingham Offspring Study. Design and preliminary data. Prev Med 4:518-25.
36. Osgood D, Corella D, Demissie S, Cupples L A, Wilson P W, Meigs J B, Schaefer E J, Coltell O, Ordovas J M (2003) Genetic variation at the scavenger receptor class B type 1 gene locus determines plasma lipoprotein concentrations and particle size and interacts with type 2 diabetes: the Framingham study. J Clin Endocrinol Metab 88:2869-79.
37. Rimm E B, Giovannucci E L, Stampfer M J, Colditz G A, Litin L B, Willett W C (1992) Reproducibility and validity of an expanded self-administered semiquantitative food frequency questionnaire among male health professionals. Am. J. Epidemiol 135:1114-1126.
38. Henneman P, Schaap F G, Havekes L M, Rensen P C, Frants R R, van Tol A, Hattori H, Smelt A H, van Dijk K W (2006) Plasma apoAV levels are markedly elevated in severe hypertriglyceridemia and positively correlated with the APOA5 S19W polymorphism. Atherosclerosis (in press).
39. KIos K L, Hamon S, Clark A G, Boerwinkle E, Liu K, Sing C F (2005) APOA5 polymorphisms influence plasma triglycerides in young, healthy African Americans and whites of the CARDIA Study. J Lipid Res 46:564-71.
40. Hubacek J A, Skodova Z, Adamkova V, Lanska V, Poledne R (2004) The influence of APOAV polymorphisms (T −1131>C and S19>W) on plasma triglyceride levels and risk of myocardial infarction. Clin Genet 65:126-30.
41. Lee K W, Ayyobi A F, Frohlich J J, Hill J S (2004) APOA5 gene polymorphism modulates levels of triglyceride, HDL cholesterol and FERHDL but is not a risk factor for coronary artery disease. Atherosclerosis 176: 165-72.
42. Aberle J, Evans D, Beil F U, Seedorf U (2005) A polymorphism in the apolipoprotein A5 gene is associated with weight loss after short-term diet. Clin Genet 68:1524.
43. Martin S, Nicaud V, Humphries S E, Talmud P J; EARS group (2003) Contribution of APOA5 gene variants to plasma triglyceride determination and to the response to both fat and glucose tolerance challenges. Biochim Biophys Acta 1637:217-25.
44. Martinelli N, Trabetti E, Bassi A, Girelli D, Friso S, PizzoloF, Sandri M, Malerba G, Pignatti P F, Corrocher R, Olivieri O (2006) The −1131 T>C and S19W APOA5 gene polymorphisms are associated with high levels of triglycerides and apolipoprotein C-III, but not with coronary artery disease: an angiographic study. Atherosclerosis (in press).
45. Liu-H, Zhang S, Lin J, Li H, Huang A, Xiao C, Li X, Su Z, Wang C, Nebert D W, Zhou B, Zheng K, Shi J, Li G, Huang D (2005) Association between DNA variant sites in the apolipoprotein A5 gene and coronary heart disease in Chinese. Metabolism 54:568-72.
46. Ruiz-Narvaez E A, Yang Y, Nakanishi Y, Kirchdorfer J, Campos H (2005) APOC3/A5 haplotypes, lipid levels, and risk of myocardial infarction in the Central Valley of Costa Rica. J Lipid Res 46:2605-13.
47. Szalai C, Keszei M, Duba J, Prohaszka Z, Kozma G T, Csaszar A, Balogh S, Almassy Z, Fust G, Czinner A (2004) Polymorphism in the promoter region of the apolipoprotein A5 gene is associated with an increased susceptibility for coronary artery disease. Atherosclerosis 173:109-14.

We claim:

1. A method for directing a diet to an individual in need thereof to allow the individual to maintain or reduce weight, manage diabetes or accommodate a food allergy, the method comprising determining whether the individual carries APOA5 −1131T and/or C allele or both or any allele in chromosome 11 that is in a tight linkage disequilibrium with said alleles, wherein if the individual is a homozygote for APOA5 −1131T allele or an allele in tight linkage disequilibrium with APOA5 −1131T allele, the individual is directed to a diet comprising a total fat content below 30% of total calorie intake and/or amount of monounsaturated fatty acids under about 11% of total calorie intake.

2. The method of claim 1, wherein one determines whether the individual carries at least one APOA5 −1131C allele or any allele in a tight linkage disequilibrium with said allele, and if the individual does not carry at least one APOA5 −113° C. allele or any allele in a tight linkage disequilibrium with said allele, the individual is directed to a diet comprising a total fat content below 30% of total calorie intake and/or amount of monounsaturated fatty acids under about 11% of total calorie intake.

3. The method of claim 1, wherein the individual is Caucasian.

4. The method of claim 1, wherein the individual is African American.

5. A kit for assisting an individual in determining whether a diet with total fat content 30% or more of total daily calorie intake or a diet with total monounsaturated fatty acid content of about 11% or more is suitable for the individual, the kit comprising a means to obtain a biological sample comprising nucleic acids, a packaging material for sending the biological material to be analyzed by a third party, optionally a return envelope for the third party to send a result of a to the individual or a requesting party, and an instruction leaflet which indicates that if the individual is a homozygote for the APOA5 −1131T allele or any acronym thereof, a diet with total fat content 30% or more of total daily calorie intake or a diet with total monounsaturated fatty acid content of about 11% or more is not suitable for the individual and if the individual carries one or two APOA5 −1131C alleles, the diet may be suitable for the individual.

6. The kit of claim 5, wherein the kit further indicates that it is useful for individuals Caucasian and African American.
